(19) United States
(12) Patent Application Publication
(10) Pub. No.: US 2025/0207094 A1
(43) Pub. Date: Jun. 26, 2025

(54) SYSTEMS AND METHODS FOR UPREGULATION OF LOW-DENSITY LIPOPROTEIN RECEPTOR (LDL-R) EXPRESSION

(71) Applicant: Kite Pharma, Inc., Santa Monica, CA (US)

(72) Inventors: Maolu Li, Hacienda Heights, CA (US); Kaiyuan Jiang, King Of Prussia, PA (US); Thanh Nguyen Yip, San Francisco, CA (US); Andrew James Sattler, Thousand Oaks, CA (US)

(21) Appl. No.: 18/987,823

(22) Filed: Dec. 19, 2024

Related U.S. Application Data
(60) Provisional application No. 63/613,503, filed on Dec. 21, 2023.

(51) Int. Cl. C12N 5/0783; A61K 40/11; A61K 40/31; A61K 40/42; C07K 14/705; C12N 5/00; C12N 15/86

(52) U.S. Cl. CPC C12N 5/0636; A61K 40/11; A61K 40/31; A61K 40/4202; C07K 14/705; C12N 5/0018; C12N 15/86; C12N 2501/2302; C12N 2523/00; C12N 2740/10043; C12N 2740/15043

(57) ABSTRACT

The present disclosure describes technologies for improving a T cell manufacturing process. Specifically, upregulating low-density lipoprotein receptor (LDL-R) expression on a T cell surface. The described technologies are particularly suitable in manufacturing processes using of non-activated T cells in a transduction step.

### CROSS-REFERENCE TO RELATED APPLICATIONS

This application claims priority to U.S. Provisional Application No. 63/613,503, filed on Dec. 21, 2023, which is incorporated herein by reference in its entirety for all purposes.

### FIELD

The present disclosure generally relates to technology for manufacturing T cells. The technology may be particularly suitable to CAR T cell manufacturing processes using non-activated T cells. The various methods of the technology may include processes for upregulating expression of low-density lipoprotein receptors (LDL-R) on a T cell's surface.

### BACKGROUND

Manufacture of cell therapy products (e.g., CAR T cell therapy products) is a relatively new field plagued with a variety of problems that require innovative solutions. Problems include an inability to maintain a high population of naïve cells, high manufacturing costs, long manufacturing times, and activation-related impurities being present in the products. Such problems arise from commonly used steps within the CAR T cell manufacturing process. For example, activation steps are often used to ensure efficient transduction, but can also lead to T cell exhaustion.

What is needed is a method of manufacturing cell therapy products (e.g., CAR T cell products) that maintains naïve T cell population, reduces manufacturing costs, shortens manufacturing times, reduces activation-related impurities in final products, and increases antitumor clinical potency while maintaining broad clinical applicability. More specifically, methods are needed to eliminate or reduce the need for activation steps in a CAR T cell manufacturing process. The technology described herein makes positive progress toward or solves these and additional problems.

### SUMMARY

In various aspects, a method for upregulating low-density lipoprotein receptor (LDL-R) expression in a population of T cells is described herein. In various embodiments, the method may include providing a population of selected T cell expressing a first quantity of membrane-bound LDL-Rs and incubating the population of T cells in a cell culture solution comprising IL-2. In various embodiments, the cultured population of T cells express a second quantity of membrane-bound LDL-Rs and the second quantity is larger than the first quantity.

In various embodiments, the cell culture solution may be 37° C. In various embodiments, the cell culture solution may remain in a 5% CO₂ environment. In various embodiments, the step of incubating may require at least 5 hours. In various embodiments, the step of incubating may require between about 20 hours to about 24 hours.

In various embodiments, the method may include providing a population of selected T cells expressing a first quantity of membrane-bound LDL-Rs and incubating the population of T cells in a cell culture solution comprising IL-2. In various embodiments, the cultured population of T cells express a second quantity of membrane-bound LDL-Rs and the second quantity is larger than the first quantity. In various embodiments, the second quantity has increased by between about 4.7-fold to about 11.0-fold compared to the first quantity. In various embodiments, the second quantity has increased by between about 4.7-fold to about 6.5-fold compared to the first quantity. In various embodiments, the second quantity has increased by between about 6.5-fold to about 11.0-fold compared to the first quantity. In various embodiments, the second quantity has increased by about 7.4-fold compared to the first quantity.

In various embodiments, the population of T cells are non-activated.

In various embodiments, method for upregulating low-density lipoprotein receptor (LDL-R) expression in a population of T cells may further comprise transducing the cultured population of T cells.

In various embodiments, a method of manufacturing CAR T cells is described herein. In various embodiments, the method of manufacturing CAR T cells may include conducting an apheresis on a patient. In various embodiments, the method of manufacturing CAR T cells may include selecting a population of T cells from the apheresis. In various embodiments, the method of manufacturing CAR T cells may include upregulating cell surface expression of low-density lipoprotein receptors (LDL-R) of the population of non-activated T cells. In various embodiments, the method of manufacturing CAR T cells may include transducing the population of T cells to generate a population of CAR T cells. In various embodiments, the method of manufacturing CAR T cells may include causing the CAR T cells to proliferate. In various embodiments, the method of manufacturing CAR T cells may include generating a cell therapy product using the CAR T cells.

In various embodiments, the method of manufacturing CAR T cells may include cryopreserving the selected population of T cells. In various embodiments, the method of manufacturing CAR T cells may include thawing the cryopreserved population of T cells. In various embodiments, the method of manufacturing CAR T cells may include washing the thawed population of T cells. In various embodiments, the method of manufacturing CAR T cells may include incubating the population of T cells in a cell culture solution comprising IL-2.

In various embodiments, the cell culture solution may be incubated at a temperature of 37° C. In various embodiments, the cell culture solution remains in an environment of 5% CO₂. In various embodiments, the incubation step requires at least 5 hours. In various embodiments, the incubation step requires between about 20 hours to about 24 hours.

In various embodiments, the population of T cells are non-activated.

In various embodiments, the transduction step uses a lentiviral vector. In various embodiments, the transduction step uses a retroviral vector.

In various embodiments, a mixture for upregulating low-density lipoprotein receptor (LDL-R) expression in a population of T cells is described. In various embodiments, the mixture may comprise a population of T cells and a plurality of IL-2 molecules.

In various embodiments, the mixture includes a temperature of 37° C. In various embodiments, the mixture may remain in an environment of 5% CO₂.

In various embodiments, the population of T cells are non-activated.

In various embodiments, a system for upregulating low-density lipoprotein receptor (LDL-R) expression in a population of T cells is described herein. In various embodiments, the system may comprise an incubator and a container for holding a mixture, wherein the container is in thermal contact with the incubator. In various embodiments, the mixture may comprise a population of T cells. In various embodiments, the mixture may comprise a plurality of IL-2 molecules.

In various embodiments, the incubator maintains a temperature of the mixture. In various embodiments, the temperature may include 37° C.

In various embodiments, the mixture may remain in an environment of 5% CO₂.

In various embodiments, the population of T cells are non-activated.

### BRIEF DESCRIPTION OF THE FIGURES

FIG. 1 illustrates a method for a T cell manufacturing process according to various embodiments.

FIG. 2 illustrates a method for upregulating low-density lipoprotein receptor (LDL-R) expression in a population of T cells in accordance with various embodiments.

FIG. 3 illustrates a method of manufacturing CAR T cells in accordance with various embodiments.

FIG. 4 illustrates a schematic diagram for a system for upregulating low-density lipoprotein receptor (LDL-R) expression in a population of T cells in accordance with various embodiments.

FIG. 5 is a table summarizing the data shown in FIGS. 6-8.

FIG. 6A is flow cytometry data showing cell-surface LDL-R expression of T cells before undergoing the method for upregulating low-density lipoprotein receptor (LDL-R) expression on cell surface.

FIGS. 6B and 6C are flow cytometry data showing cell-surface LDL-R expression on T cells having undergone the method for upregulating low-density lipoprotein receptor (LDL-R) expression on cell surface.

FIG. 7A is flow cytometry data showing cell-surface LDL-R expression of T cells before undergoing the method for upregulating low-density lipoprotein receptor (LDL-R) expression on cell surface.

FIGS. 7B and 7C are flow cytometry data showing cell-surface LDL-R expression on T cells having undergone the method for upregulating low-density lipoprotein receptor (LDL-R) expression on cell surface.

FIG. 8A is flow cytometry data showing cell-surface LDL-R expression of T cells before undergoing the method for upregulating low-density lipoprotein receptor (LDL-R) expression on cell surface.

FIGS. 8B and 8C are flow cytometry data showing cell-surface LDL-R expression on T cells having undergone the method for upregulating low-density lipoprotein receptor (LDL-R) expression on cell surface.

### DETAILED DESCRIPTION OF THE DISCLOSURE

The present disclosure provides insights and technologies useful in a CAR T cell manufacturing process among other things. More specifically, the embodiments described herein may be useful for improving transduction of non-activated T cells by upregulating low-density lipoprotein receptors (LDL-Rs) on T cell surface. In various embodiments, LDL-Rs may be a port of entry for viral vector. Being able to increase the number of ports available without an activation step may solve many CAR T cell manufacturing problems the field faces. Some of the advantages include reduced T cell exhaustion and increased percentage of naïve T cells that are generated in the process.

Embodiments of methods for upregulating low-density lipoprotein receptor (LDL-R) expression on T cell surface are described in the accompanying description and figures. In the figures, numerous specific details are set forth to provide a thorough understanding of certain embodiments. A skilled artisan will appreciate that the systems and methods described herein may be used in a variety of ways and circumstances that are not limited to what is specifically detailed. Additionally, the skilled artisan will appreciate that certain embodiments may be practiced without these specific details. Furthermore, one skilled in the art can readily appreciate that the specific sequences in which methods are presented and performed are illustrative and it is contemplated that the sequences can be varied and still remain within the spirit and scope of certain embodiments.

While the present teachings are described in conjunction with various embodiments, it is not intended that the present teachings be limited to such embodiments. On the contrary, the present teachings encompass various alternatives, modifications, and equivalents, as will be appreciated by those skilled in the art.

### I. Definitions

In order that the present disclosure may be more readily understood, certain terms are first defined. As used in this application, except as otherwise expressly provided herein, each of the following terms shall have the meaning set forth below. Additional definitions are set forth throughout the application. The headings provided herein are not limitations of the various aspects of the disclosure, which aspects should be understood by reference to the specification as a whole.

As used herein, the terms “a” and “an” are used per standard convention and mean one or more, unless context dictates otherwise.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. Alternatively, “about” or “comprising essentially of” can mean a range of up to 10% (i.e., ±10%). For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

As used herein, the term “and/or” is to be understood as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or,” as used in a phrase such as ‘A, B, and/or C” is intended to encompass each of the following aspects: A, B, and C; A, B, or C; A or C; A or B; B or C; A and C; A and B; B and C; A (alone); B (alone); and C (alone).

As used herein, the term the use of the alternative (e.g., “or”) should be understood to mean either one, both, or any combination thereof of the alternatives.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

As used herein, the term “apheresis material” may originate from blood. In various embodiments, patient/donor derived blood may undergo apheresis to generate an apheresis material. In various embodiments, the apheresis material may include leukocytes. Additional examples of an apheresis material may include platelets, white blood cells, or any constituent of blood. In more specific examples, T cells may be collected from patients for later processing (e.g., T cell modification).

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient. In various embodiments, the methods described herein may be incorporated into an autologous T cell manufacturing system and/or process.

The term “allogeneic” refers to any material derived from one individual which is then introduced to another individual of the same species, e.g., allogeneic T cell transplantation. In various embodiments, the methods described herein may be incorporated into an allogeneic T cell manufacturing system and/or process.

The term “cancer” may refer to a broad group of various diseases characterized by the uncontrolled growth of abnormal cells in the body. The cell therapy products the T cell manufacturing processes described herein are designed to generate may be used to treat cancer among other things. Cancer may mean unregulated cell division and growth results in the formation of malignant tumors that invade neighboring tissues and may also metastasize to distant parts of the body through the lymphatic system or bloodstream. A “cancer” or “cancer tissue” may include a tumor. In this application, the term cancer is synonymous with malignancy. Examples of cancers that may be treated by the methods disclosed herein include, but are not limited to, cancers of the immune system including lymphoma, leukemia, myeloma, and other leukocyte malignancies. In some embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers including those induced by asbestos, other B cell malignancies, and combinations of said cancers. In some embodiments, the cancer is multiple myeloma. In some embodiments, the cancer is NHL. The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

As used herein, the term “cell therapy product” refers to a therapy including viable cells. The viable cells may be administered to a patient. Administration may occur by injection, translation, or infusion. In various embodiments, the viable cells may include immune cells. In various embodiments, the immune cells may include T cells. In various embodiments, the T cells may include chimeric antigen receptors (CARs).

The term “genetically engineered” or “engineered” may refer to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

As used herein, the term “low-density lipoprotein (LDL)” refers to lipoprotein molecules that facilitate transfer lipids (e.g., fats) around the body in the extracellular fluid, making fats available to body cells for receptor-mediated endocytosis.

T cells of the immunotherapy may come from any source known in the art. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

As used throughout, the term “likely” refers to having a higher probability of occurring than not, or alternatively, of having a higher probability of occurring versus a predetermined control of average. By way of non-limiting example, a patient likely to experience toxicity following a cell therapy refers to that patient having a higher probability of experiencing toxicity than not. Alternatively, a patient likely to experience toxicity following a cell therapy refers to that patient having a higher statistical chance of experiencing toxicity as compared to the average occurrence of toxicity in a patient population treated with the cell therapy. One of ordinary skill in the art would recognize additional definitions in addition to the aforementioned.

As used herein, the term “low-density lipoprotein receptor (LDL-R)” refers to protein molecule typically embedded in the cell membrane. Typically, LDL-R mediates the endocytosis of cholesterol-rich low-density lipoprotein (LDL). In addition, LDL-R mediates the transfer of lentiviral particles from the extracellular fluid to the intracellular fluid of cells. In various embodiments, the N-terminal domain of LDL-R is responsible for ligand binding.

The term “lymphocyte” as used herein includes natural killer (NK) cells, T cells, or B cells. NK cells are a type of cytotoxic (cell toxic) lymphocyte that represent a major component of the innate immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). Its T cell receptors (TCR) differentiate themselves from other lymphocyte types. The thymus, a specialized organ of the immune system, is primarily responsible for the T cell's maturation. T cells can be divided in CD4+ subset (Helper T cells) and CD8+ subset (Cytotoxic T cells). Based on the level of differentiation, T cells can also be divided into 4 subsets: naïve, CM (central memory), EM (effector memory) and TEMRA (Terminally differentiated effector memory) B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

As used herein, the term “patient” means any human who is being treated for an abnormal physiological condition, such as cancer or has been formally diagnosed with a disorder, those without formally recognized disorders, those receiving medical attention, those at risk of developing the disorders, etc. The terms “subject” and “patient” are used interchangeably herein and include both human and non-human animal subjects.

As used herein, the term “pre-treatment” may refer to a cell or population of cells that have not undergone one or more of the methods or method steps described herein for upregulating expression of LDL-R. In various embodiments, the cell or population of cells may include a T cell or a population of T cells.

As used herein, the term “post-treatment” may refer to a cell or population of cells that have undergone one or more of the methods or method steps described herein for upregulating expression of LDL-R. In various embodiments, the cell or population of cells may include a T cell or a population of T cells.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. Similarly, the term “increasing” indicates any change that is higher than the original value. “Increasing,” “higher,” and “lower” are relative terms, requiring a comparison between pre- and post-measurements and/or between reference standards.

As used herein, the term “selected T cells” refers to T cells that have undergone a T cell selection process (e.g., see section III. a herein). In various embodiments, selected T cells may refer to selected T cells that have been cryopreserved and may be made available for further processing after undergoing a thawing protocol.

As used herein, the term “T cell exhaustion” may refer to a state T cells can enter during chronic stimulation. T cell exhaustion may lead to loss of effector activity, loss of proliferation capacity, and other dysfunctions. The methods described herein may reduce T cell exhaustion by eliminating the need for a T cell activation step in a T cell manufacturing process.

The terms “transduction” and “transduced” may refer to the process whereby foreign DNA is introduced into a cell via viral vector (see Hartl and Jones (1997) “Genetics: Principles and Analysis,” 4^{th} ed, Jones & Bartlett). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In another embodiment, “treatment” or “treating” includes a complete remission. In various embodiments, the methods of upregulating low-density lipoprotein receptor (LDL-R) expression in cells (e.g., T cells) can cause improved treatment outcomes. Non-limiting examples may include may increasing the overall viability of cells used for immunotherapy treatment, the quantity of transduced cells, the percentage of cells retaining their naïve state. Overall quality of a cell therapy product used for treatment is improved using the LDL-R upregulation of expression methods described herein.

The disclosure further provides diagnostic, prognostic and therapeutic methods, which are based, at least in part, on determination of the expression level of a gene of interest identified herein.

It is understood that the technologies described herein may be used alone or in combination with other technologies. For example, the methods for upregulation expression of low-density lipoprotein receptors (LDL-Rs) may be incorporated into a broader manufacturing workflow for generating cell therapy products that may be used to treat a patient.

### II. Overview

The methods described herein are intended to improve methods of manufacturing cell therapy products (e.g., CAR T cell products) by reducing manufacturing costs, shortening manufacturing times, reducing activation-related impurities in final products, and increasing antitumor clinical potency while maintaining broad clinical applicability. The methods described herein cause the upregulation of low-density lipoprotein receptors (LDL-Rs) on the surface of non-activated T cells which can lead to increased transduction efficiencies.

It will be appreciated that chimeric antigen receptors (CARs or CAR-Ts) are, and T cell receptors (TCRs) may, be genetically engineered receptors. These engineered receptors may be readily inserted into and expressed by immune cells, including T cells in accordance with techniques known in the art. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell. The methods for upregulating LDL-Rs described herein may be well suited for the generation of CAR T cells.

Low-density lipoprotein receptor (LDL-R) serves as the main receptor of entry of lentiviral particles that are pseudotyped with the vesicular stomatitis virus g-glycoprotein (VSV-G), and therefore LDL-R expression is a key factor for efficient lentiviral vector transduction.

Often, transducing non-activated T-cells is found to be inefficient and one attributing factor is the low expression of LDL-R on non-activated T-cells (“Rapid manufacturing of non-activated potent CAR T cells,” Nat. Biomed. Eng., 2022, 6, 118-128 the disclosure of which is hereby incorporated by reference in its entirety for all purposes). LDL-R expression is associated with the metabolic state of the cells and therefore LDL-R upregulation can be achieved by modifying the culture conditions, such as cholesterol restriction achieved by a brief serum starvation, as described in “Rapid manufacturing of non-activated potent CAR T cells,” Nat. Biomed. Eng., 2022, 6, 118-128. The method for upregulation of low-density lipoprotein receptor (LDL-R) expression in T cells described herein does not require serum starvation. In the methods described herein, non-activated T-cells were cultured in a cell culture solution, comprising the cytokine IL-2, overnight (e.g., at least 5 hours or 20-24 hours) at 37° C. and 5% CO₂ to upregulate LDL-R expression on the cell surface to create the possibility of more effective vector entry and thus potentially enhance transduction efficiency.

The methods described herein for the upregulation of low-density lipoprotein receptor (LDL-R) expression on the surface of T cells are suitable for T cells that are non-activated and will undergo viral transduction. Transducing non-activated T cells generates higher populations of naïve cells which may lead to enhanced cell proliferation, prolonged CAR T cell persistence, improved clinical efficacy at lower doses, and an improved safety profile for the patient.

### III. Exemplary T Cell Manufacturing Process

Provided herein are methods for manufacturing engineered human lymphocytes for cell therapy products. Cells of interest may be isolated via positive or negative selection using density gradient, magnetic bead, or acoustic forces to obtain a mass of enriched/selected cells ready for activation via environmental pressures or antibody co-stimulation, the latter which is envisioned to be done sequentially or concurrently with isolation using antibody conjugated or physical coated beads, labels, surfaces, or particles bound to target cells. Cells may be washed via centrifugation or buffer exchange and transfected or transduced through physical co-positioning of cell and vector (RVV, LVV) or DNA/RNA capsule. Transfection and/or transduction may be designed to be done with or without pre reagent coating of culture surfaces. The transduced cells may be pre-washed or directly enter into an expansion step utilizing various batch, batch-fed, perfusion, and solera methods to obtain a sufficient number of cells to meet dose. Final formulation may be achieved through addition of cryoprotectant reagents and buffers to the cells at a specified ratio via buffer exchange, acoustic separation, centrifugation, gravitation, pumping, or syringe fluid handling techniques.

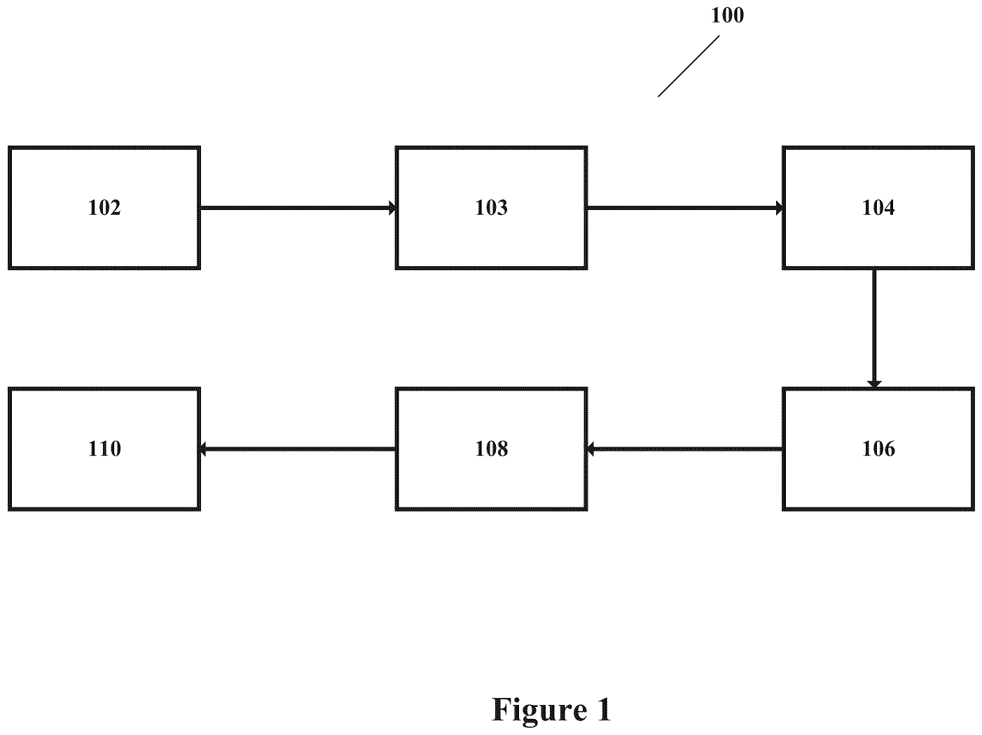

FIG. 1 illustrates a method for a T cell manufacturing process 100 according to various embodiments. The steps in the T cell manufacturing process 100 may include one or more of cell wash and selection 102, upregulation of low-density lipoprotein receptor (LDL-R) expression in T cells 103, transfection or transduction 104, expansion 106, depletion 108, and harvest wash and final formulation 110.

### a) T Cell Selection

The T cell manufacturing process 100 includes a T cell selection process 102, in accordance with various embodiments. In various embodiments, the T cell selection process 102 may include CD4+/CD8+ T cell enrichment carried out via magnetic bead or acoustic selection isolation with a T cell recovery of 30-80% (relative to incoming apheresis T cell composition) at a T cell purity of more than 85% and viability of typically above 90%.

### i) Upregulation of Low-Density Lipoprotein Receptor (LDL-R) Expression in T Cells

In various embodiments, the T cell manufacturing process 100 may include a process for upregulating low-density lipoprotein receptor (LDL-R) expression in T cells 103. See Section IV. Exemplary Methods for Upregulating Low-Density Lipoprotein Receptor (LDL-R) Expression in a Population of T Cells for additional description.

### b) Transfection or Transduction

The T cell manufacturing process 100 includes an transfection and/or transduction 104, in accordance with various embodiments. In various embodiments, the electroporation and/or transduction step includes transfecting the concentrated cells (15-300 e6/mL) with genetic or non-genetic material (e.g. DNA or RNA encoding ZFN or CRISPR or TALENs) to affect the desired gene modifications (gene knockout or additions). Post-electroporation, the cells may be washed, buffer exchanged, or diluted to minimize exposure to the electroporation buffer during transduction.

In various embodiments, the T cells may be transduced with construct-encoding lentiviral vectors (LVV) or retroviral vectors (RVV) using enhancing reagents at optimized conditions (retronectin, protamine sulfate, polybrene, or vectofusin-1) or enhancer-free physical co-localization viral vector-based gene delivery methods at a cell to vector ratio designed to achieve desired transduction efficiencies and genomic integration. The volume of viral vector is controlled at a target multiplicity of infection (transducing viral particle units per cell) and incorporated into the transduction system or the culture system. The transduction seed density may typically be between 1-5 e6 cells/mL (to achieve the desired particle per cell unit ratio) and may last from 1 hour to 72 hours at temperature ranges from 15° C. to 37° C.

### c) Expansion

The T cell manufacturing process 100 includes an expansion step 106, in accordance with various embodiments. In various embodiments, following gene editing and transduction, the cells may be expanded in static, shake flasks, rocking wave bioreactors, or stirred tank bioreactors to achieve the desired dose.

### d) Depletion

The T cell manufacturing process 100 may include a depletion step 108, in accordance with various embodiments. In various embodiment, after expansion, the expanded cells may be washed via centrifugation, buffer exchange, or acoustic separation to achieve a desired cell concentration of 50-300 e9 cells/mL in 200-500 mL of media. The concentration rate is designed to maintain throughput targets across the unit operations. Depletion may then be performed via a negative selection stepwise isolation step to deplete the unedited TCRab+ cells for improved product purity and quality.

### e) Final Formulation

The T cell manufacturing process 100 includes a final formulation step 110, in accordance with various embodiments. In various embodiments, a final formulation step may occur after a sufficient number of target engineered cells are achieved to meet a dose specification. Formulation involves a harvest wash step using the following options: centrifugation resuspension via Cytiva Sepax Culture Wash or Sefia FlexCell, perfusion dilution via Cytiva Xuri WAVE system or Applikon Biosep cell retention, or buffer exchange inertial flow fluid dynamics. After the wash step, a dose specific post-wash volume is combined with cryoprotectant reagents and buffers at a specified ratio in a closed vessel. For final formulation, the step may occur in using the Terumo FINIA system, the buffer exchange inertial flow device, or traditional manual methods using gravitational, pump, or syringe fluid handling techniques. The final product bags are cryopreserved for storage, shipment, and later use.

### IV. Exemplary Methods for Upregulating Low-Density Lipoprotein Receptor (LDL-R) Expression in a Population of T Cells

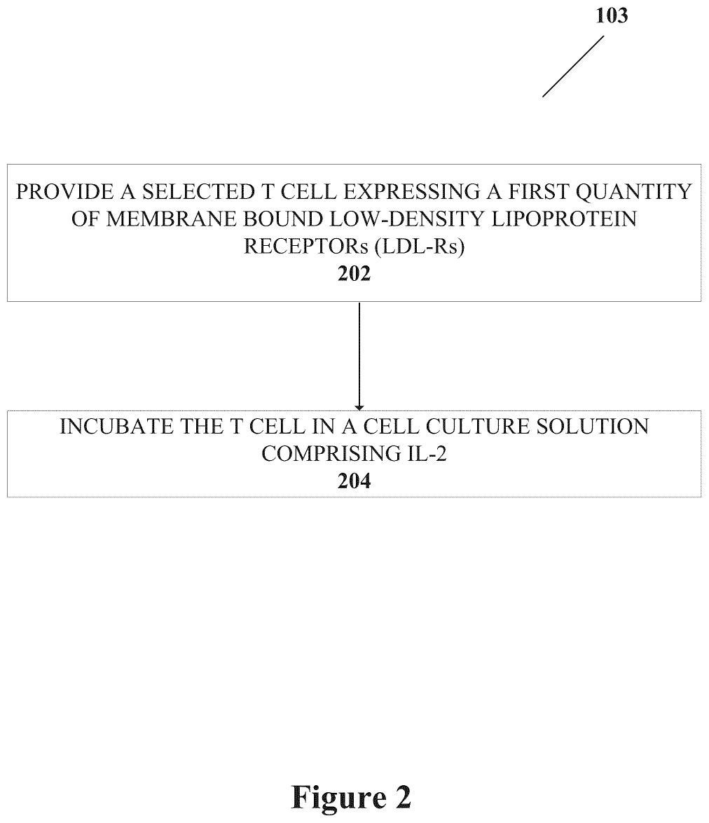

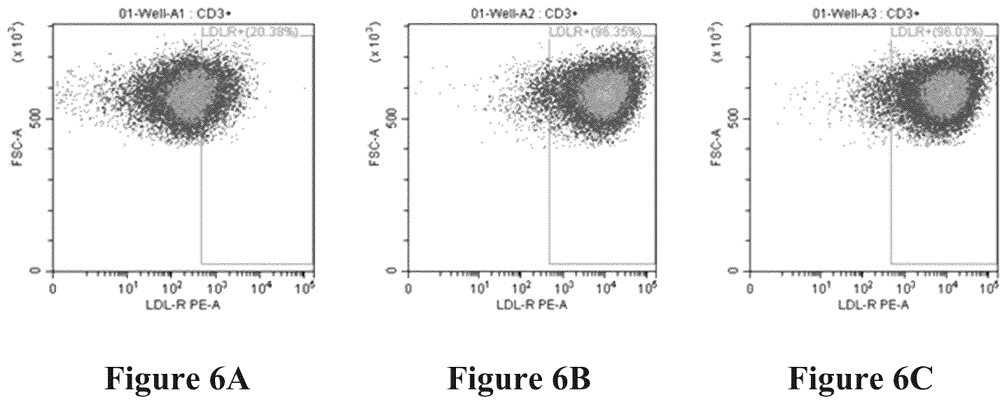

FIG. 2 illustrates a method for upregulating low-density lipoprotein receptor (LDL-R) expression in a population of T cells 103 in accordance with various embodiments. The method for upregulating low-density lipoprotein receptor (LDL-R) expression in a population of T cells may comprise providing a population of selected T cell expressing a first quantity of membrane-bound LDL-Rs 202 and incubating the population of T cells in a cell culture solution comprising IL-2 204. In various embodiments, the cultured population of T cells express a second quantity of membrane-bound LDL-Rs, wherein the second quantity is larger than the first quantity. The method detailed herein leads to a several-fold increase in the quantity of LDL-R expressing T cells within the overall population of T cells. For example, incubating cells in media comprising IL-2 at a specified temperature for a specified time duration may lead to the increase in LDL-R expression as shown in FIGS. 6B, 7B, and 8B and 6C, 7C, and 8C as compared to FIGS. 6A, 7A, and 8A respectively (see Examples).

In various embodiments, the cell culture solution may be maintained at a stable temperature. In alternative embodiments, the temperature may be varied over the course of an incubation cycle. In various embodiments, an effective temperature for the cell culture solution may include 37° C.

In various embodiments, the cell culture solution may be maintained at 30° C., 31° C., 32° C., 33° C., 34° C., 35° C., 36° C., 37° C., 38° C., 39° C., 40° C., 41° C., 42° C., 43° C., 44° C., 45° C. or any range therebetween.

In various embodiments, gases may be introduced into the cell culture solution. In various embodiments, cell culture solution may comprise CO₂. In various embodiments, cell culture solution may comprise O₂. In various embodiments, cell culture solution may comprise N₂.

In various embodiments, the concentration of CO₂ in the cell culture solution may be at least 0, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 15%, 20%, 25%, 30%, 35%, 40%, 45%, 50%, 60%, 70%, 80%, 90%, 100% or a range therein. In various embodiments, the concentration of CO₂ in the cell culture solution may be about 5%.

In various embodiments, the step of incubating requires at least 1, 1.5, 2, 2.5, 3, 3.5, 4, 4.5, 5, 6, 7, 8, 9, 10, 11, 12, 13, 14, 15, 16, 17, 18, 19, 20, 21, 22, 23, or 24 hours. In various embodiments, the step of incubating requires at least 5 hours. In various embodiments, the step of incubating requires between about 20 hours to about 24 hours.

In various embodiments, different components may be added to the cell culture solution to change the character. For example, agents may be added to adjust the pH and/or tonicity. In various embodiments, a cell culture medium may be added to the cell culture solution.

In various embodiments, the methods described herein significantly increase low-density lipoprotein receptor (LDL-R) expression in a population of T cells. In various embodiments, the percentage increase may be 25%, 50%, 75%, 100%, 150%, 200%, 300%, 400%, 500%, 600%, 700%, 800%, 900%, 1000%, 1100%, 1200%, 1300%, 1400%, 1500%, 1600%, 1700%, 1800%, 1900%, 2000%, 3000%, 4000% or any range therebetween.

In various embodiments, the fold increase may range from 4.7-fold to about 11.0-fold post-treatment. In various embodiments, the fold increase may range from 4.7-fold to about 6.5-fold post-treatment. In various embodiments, the fold increase may range from 6.5-fold to about 11.0-fold post-treatment. In various embodiments, the fold increase may about 7.4-fold.

T cell activation may increase LDL-R expression on the surfaces of T cells. However, activation has several disadvantages described herein and elsewhere. Specifically, T cell activation can cause T cell exhaustion. As such, the methods described herein may act as an alternative way to increase LDL-R expression without the disadvantages of activation. As such, in various embodiments, the methods described herein may be ideally suited for non-activated T cells and the T cells used herein may be non-activated.

In various embodiments, the population of T cells having undergone one or more of the methods for upregulating LDL-R expression herein may be transduced at higher efficacies than T cells not having undergone a method for upregulating LDL-R expression described herein.

In various embodiments, the process 103 may include providing selected T cells 202 and culturing non-activated T cells in a solution comprising IL-2. In various embodiments, the selected T cells express a first quantity of membrane-bound LDL-Rs prior to incubation and a second quantity of membrane-bound LDL-Rs after incubation. In various embodiments, the second quantity is larger because of contents of the cell culture and the procedure performed as described herein.

In various embodiments, the selected T cells 202 may have undergone a cell selection process described herein or elsewhere. In various embodiments, the T cells may have undergone a cryopreservation step after the selection process. In embodiments where the T cells have undergone a cryopreservation step, the method for upregulating low-density lipoprotein receptor (LDL-R) expression may further comprise thawing the selected T cells.

In various embodiments, the method may comprise washing the selected T cells. A variety of washing protocols are detailed herein and elsewhere and may be incorporated into the method.

In various embodiments, the T cells become chimeric antigen receptor T cells (CAR T cells) after a transduction step is performed.

### V. Exemplary Methods of Manufacturing CAR T Cells

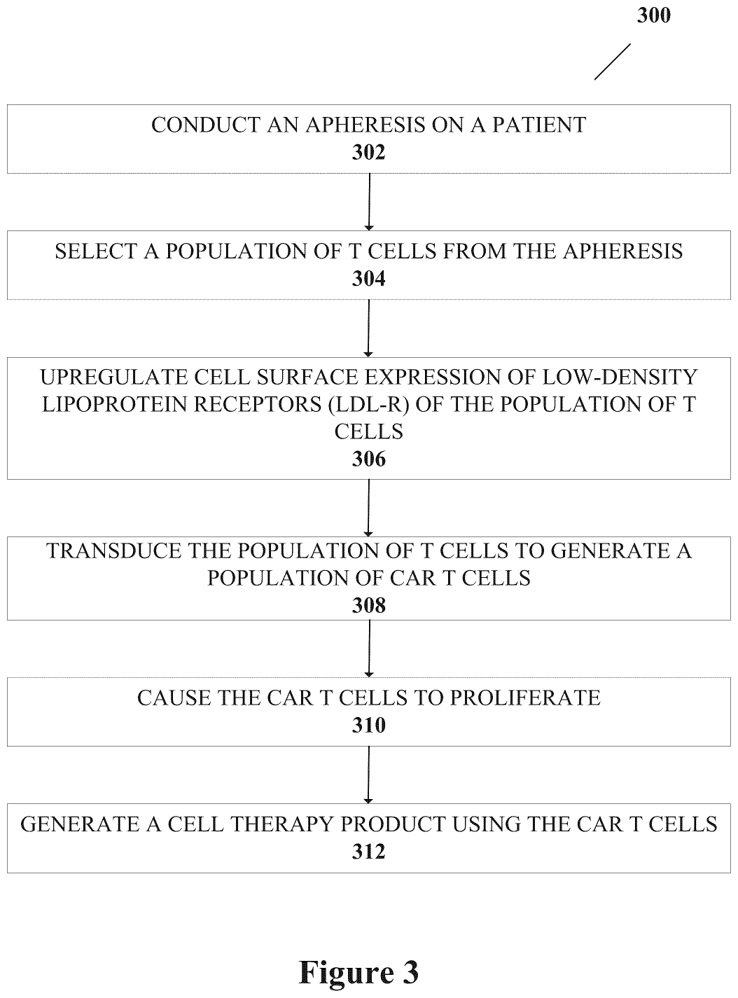

FIG. 3 illustrates a method of manufacturing CAR T cells 300 in accordance with various embodiments. In various embodiments, the steps may comprise methods described herein (e.g., Section III. Exemplary T Cell Manufacturing Process and elsewhere).

In various embodiments, the method of manufacturing CAR T cells may include conducting an apheresis on a patient 302. In various embodiments, an apheresis material may include red blood cells, white blood cells, platelets, plasma, and/or population of T cells.

In various embodiments, the method of manufacturing CAR T cells may include selecting a population of T cells from the apheresis 304.

In various embodiments, the method of manufacturing CAR T cells may include upregulating cell surface expression of low-density lipoprotein receptors (LDL-R) of the population of T cells 306.

In various embodiments, the method of manufacturing CAR T cells may include transducing the population of T cells to generate a population of CAR T cells 308.

In various embodiments, the method of manufacturing CAR T cells may include causing the CAR T cells to proliferate 310.

In various embodiments, the method of manufacturing CAR T cells may include generating a cell therapy product using the CAR T cells 312.

Various optional steps may be included in the method of manufacturing CAR T cells, including cryopreserving the selected population of T cells. In various embodiments, the method of manufacturing CAR T cells may include thawing the cryopreserved population of T cells. In various embodiments, the method of manufacturing CAR T cells may include washing the thawed population of T cells.

In various embodiments, the incubation step may include use of timepoints, ingredients, and other factors that may contribute to upregulating cell surface expression of low-density lipoprotein receptors (LDL-R) of populations of T cells. For example, the step of upregulating cell surface expression of low-density lipoprotein receptors (LDL-R) of the population of T cells may further comprise incubating the population of T cells in a cell culture solution comprising IL-2.

In various embodiments, the cell culture solution may be incubated at a temperature of 37° C. In various embodiments, the cell culture solution may be maintained at 30° C., 31° C., 32° C., 33° C., 34° C., 35° C., 36° C., 37° C., 38° C., 39° C., 40° C., 41° C., 42° C., 43° C., 44° C., 45° C. or any range therebetween.

In various embodiments, the concentration of CO₂ in the cell culture solution may be at least 0, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 15%, 20%, 25%, 30%, 35%, 40%, 45%, 50%, 60%, 70%, 80%, 90%, 100% or a range therein. In various embodiments, the concentration of CO₂ in the cell culture solution may be about 5%.

In various embodiments, the step of incubating requires at least 1, 1.5, 2, 2.5, 3, 3.5, 4, 4.5, 5, 6, 7, 8, 9, 10, 11, 12, 13, 14, 15, 16, 17, 18, 19, 20, 21, 22, 23, or 24 hours. In various embodiments, the step of incubating requires at least 5 hours. In various embodiments, the step of incubating requires between about 20 hours to about 24 hours.

In various embodiments, different components may be added to the cell culture solution to change the character. For example, agents may be added to adjust the pH and/or tonicity. In various embodiments, a cell culture medium may be added to the cell culture solution.

In various embodiments, the population of T cells are non-activated. In various embodiments, the transduction step may use a lentiviral vector. In various embodiments, the transduction step may use a retroviral vector.

### VI. Exemplary Mixtures for Upregulating Low-Density Lipoprotein Receptor (LDL-R) Expression in a Population of T Cells

In various embodiments, a mixture for upregulating low-density lipoprotein receptor (LDL-R) expression in a population of T cells is described. In various embodiments, the mixture may comprise a buffer and one or more agents used to adapt the mixture to be hospitable for T cells.

In various embodiments, the mixture may comprise a population of T cells. T cells may be sourced from a variety of different places (e.g., healthy or patient donor).

In various embodiments, the mixture may comprise a plurality of IL-2 molecules. IL-2 molecules may be sourced from a variety of commercial sources (e.g., Thermo Fisher Scientific, Inc.—Human IL-2 Recombinant Protein, PeproTech®, Cat #200-02-1MG; Mouse IL-2, Animal-Free Recombinant Protein, PeproTech®, Cat #AF-212-12-1MG; Human IL-2 Recombinant Protein, Cat #PHC0021, etc.)

In various embodiments, the cell culture solution may be incubated at a temperature of 37° C. In various embodiments, the cell culture solution may be maintained at 30° C., 31° C., 32° C., 33° C., 34° C., 35° C., 36° C., 37° C., 38° C., 39° C., 40° C., 41° C., 42° C., 43° C., 44° C., 45° C. or any range therebetween.

In various embodiments, the concentration of CO₂ in the cell culture solution may be at least 0, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 15%, 20%, 25%, 30%, 35%, 40%, 45%, 50%, 60%, 70%, 80%, 90%, 100% or a range therein. In various embodiments, the concentration of CO₂ in the cell culture solution may be about 5%.

In various embodiments, the population of T cells are non-activated.

### VI. Exemplary Systems for Upregulating Low-Density Lipoprotein Receptor (LDL-R) Expression in a Population of T Cells

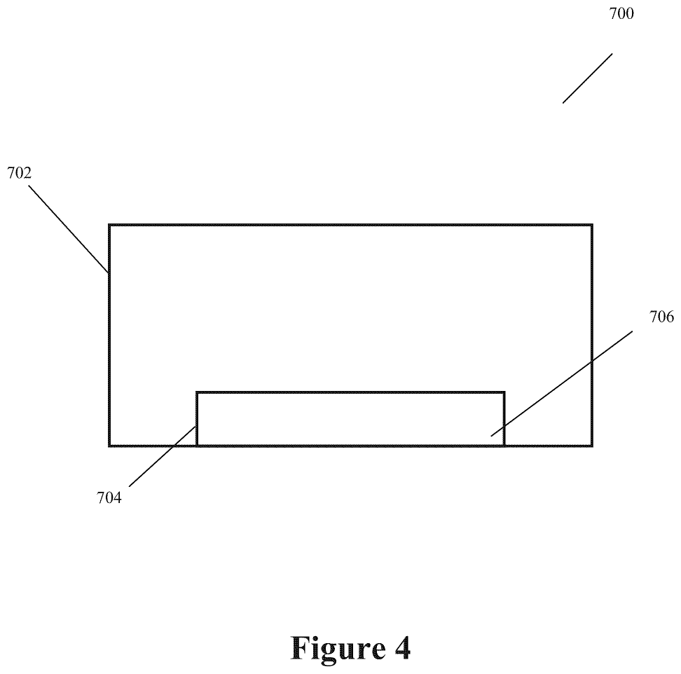

FIG. 4 illustrates a schematic diagram for a system for upregulating low-density lipoprotein receptor (LDL-R) expression in a population of T cells 700 in accordance with various embodiments. In various embodiments, the system may include a container 704 in thermal contact with an incubator 702 or a component thereof. In various embodiments, the container 704 may hold a mixture 706.

There are a variety of commercially available incubators 702. Non-limiting examples may include heating blocks, temperature-controlled spaces, etc.

There are a variety of commercially available containers 704. Non-limiting examples may include vials, beakers, tubes, culture bags, plates, etc. In various embodiments, the incubator 702 maintains a temperature of the mixture 706, wherein the temperature is 37° C. In various embodiments, the incubator 702 maintains a temperature of the mixture of about 37° C. In various embodiments, the incubator 702 maintains a temperature of the mixture of about 30° C., 31° C., 32° C., 33° C., 34° C., 35° C., 36° C., 37° C., 38° C., 39° C., 40° C., 41° C., 42° C., 43° C., 44° C., 45° C. or any range there between.

In various embodiments, the mixture 706 comprises a population of T cells and a plurality of IL-2 molecules.

In various embodiments, the concentration of CO₂ in the cell culture solution may be at least 0, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 15%, 20%, 25%, 30%, 35%, 40%, 45%, 50%, 60%, 70%, 80%, 90%, 100% or a range therein. In various embodiments, the concentration of CO₂ in the cell culture solution may be about 5%.

In various embodiments, the population of T cells are non-activated.

### VIII. Examples: T Cells Comprising Upregulated Low-Density Lipoprotein Receptor (LDL-R) Expression

Three studies were completed, demonstrating an increase in low-density lipoprotein (LDL-R) expression post-treatment using the method detailed herein and below. Specifically, these examples illustrate upregulation of low-density lipoprotein receptor (LDL-R) on membrane surfaces of T cells after application of the method.

Non-activated T cells (CD3+) were sourced from a frozen stock of T cells that had previously undergone a selection process. The selected T cells were thawed using a commercially available warming device or incubator. T cells were then washed using a Sepax C-Pro™ cell processing instrument. The T cells did not undergo an activation step and as such are “non-activated T cells.” All the flow cytometry data was collected using a CytoFLEX™ flow cytometer produced by Beckman Coulter™. A NeucleoCounter NC-200™ was used in the cell counting steps.

Commercially available antibody/fluorophores (e.g., Human LDL-R PE-conjugated antibody (FAB2148P) from R&D Systems) were used to detect LDL-R expression on T cell surfaces.

The x-axis of the plots shown in FIGS. 6-8 is the LDL-R expression reading. The y-axis of the plots shown in FIGS. 6-8 is the forward scatter area (FSC-A). FSC-A is proportional to cell-surface area or size.

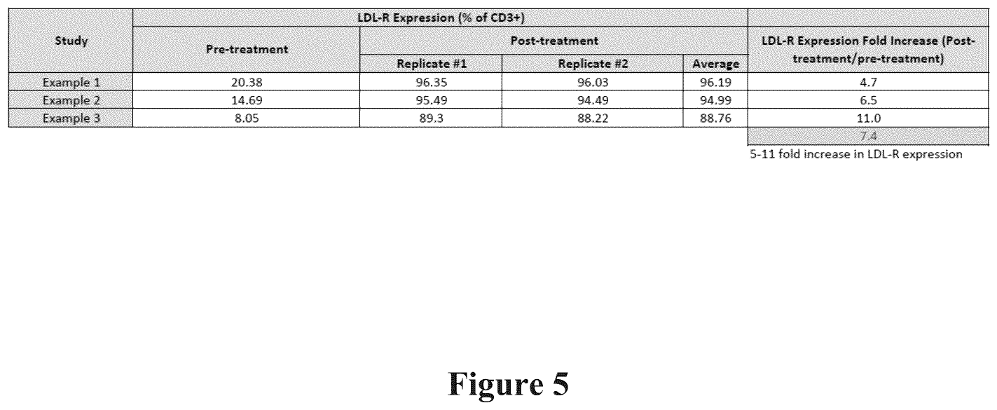

FIG. 5 is a table summarizing the data shown in FIGS. 6-8. The first column entitled “Study” lists Examples 1-3 which correspond to the headings below. The percentage of cells showing detectable levels of cell surface LDL-R expression for non-activated T cells in a pre-treatment population for Examples 1, 2, and 3 was 20.38%, 14.69%, and 8.05% respectively.

The percentage of cells showing detectable levels of cells surface LDL-R expression in a post-treatment population in Example 1, Replicate #1 was 96.35%. The percentage of cells showing detectable levels of cells surface LDL-R expression in a post-treatment population in Example 1, Replicate #2 was 96.03%. The average of Replicate #1 and Replicate #2 is 96.19%. LDL-R expression levels between pre-treatment and post-treatment is a 4.7-fold increase.

The percentage of cells showing detectable levels of cells surface LDL-R expression in a post-treatment population in Example 2, Replicate #1 was 95.49%. The percentage of cells showing detectable levels of cells surface LDL-R expression in a post-treatment population in Example 1, Replicate #2 was 94.49%. The average of Replicate #1 and Replicate #2 is 94.99%. LDL-R expression levels between pre-treatment and post-treatment is a 6.5-fold increase.

The percentage of cells showing detectable levels of cells surface LDL-R expression in a post-treatment population in Example 3, Replicate #1 was 89.3%. The percentage of cells showing detectable levels of cells surface LDL-R expression in a post-treatment population in Example 1, Replicate #2 was 88.22%. The average of Replicate #1 and Replicate #2 is 88.76%. LDL-R expression levels between pre-treatment and post-treatment is an 11-fold increase.

The average fold increase between pre-treatment and post-treatment from the three Examples is 7.4.

### a) Example 1

FIG. 6A is flow cytometry data showing cell-surface LDL-R expression of T cells before (Day 0) undergoing the method for upregulating low-density lipoprotein receptor (LDL-R) expression described herein and above.

The percentage of cells showing detectable levels of cell surface LDL-R expression for non-activated T cells in a pre-treatment population was 20.38%. Data was collected on Day 0.

FIGS. 6B and 6C are flow cytometry data showing cell-surface LDL-R expression on T cells having undergone (Day 1) the method for upregulating low-density lipoprotein receptor (LDL-R) expression on the cell surface described herein. The cell populations shown in FIGS. 6B and 6C have shifted to the right-hand side of their plots relative to the population shown in FIG. 6A which indicates an increase in LDL-R expression levels.

The percentage of cells showing detectable levels of cells surface LDL-R expression in a post-treatment population in Example 1, Replicate #1 was 96.35%. The percentage of cells showing detectable levels of cells surface LDL-R expression in a post-treatment population in Example 1, Replicate #2 was 96.03%. Data was collected on Day 1.

### b) Example 2

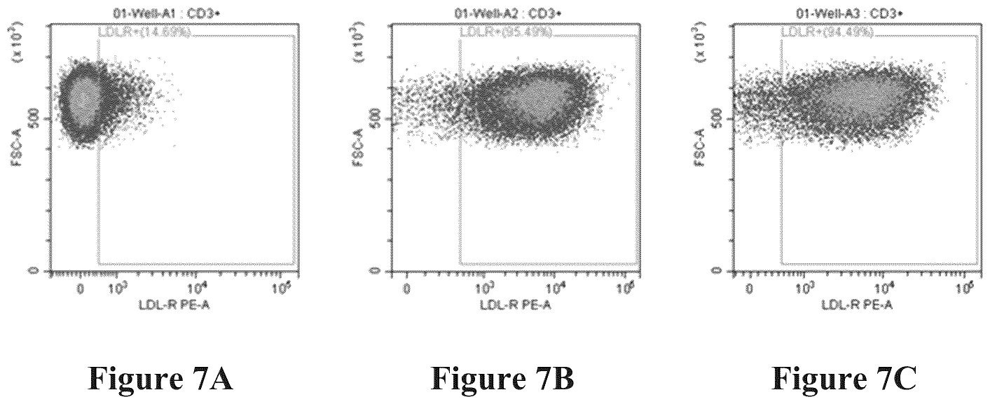

FIG. 7A is flow cytometry data showing cell-surface LDL-R expression of T cells before (Day 0) undergoing the method for upregulating low-density lipoprotein receptor (LDL-R) expression described herein and above.

The percentage of cells showing detectable levels of cell surface LDL-R expression for non-activated T cells in a pre-treatment population was 14.69%. Data was collected on Day 0.

FIGS. 7B and 7C are flow cytometry data showing cell-surface LDL-R expression on T cells having undergone (Day 1) the method for upregulating low-density lipoprotein receptor (LDL-R) expression on the cell surface described herein. The cell populations shown in FIGS. 7B and 7C have shifted to the right-hand side of their plots relative to the population shown in FIG. 7A which indicates an increase in LDL-R expression levels.

The percentage of cells showing detectable levels of cells surface LDL-R expression in a post-treatment population in Example 2, Replicate #1 was 95.49%. The percentage of cells showing detectable levels of cells surface LDL-R expression in a post-treatment population in Example 2, Replicate #2 was 94.49%. Data was collected on Day 1.

### c) Example 3

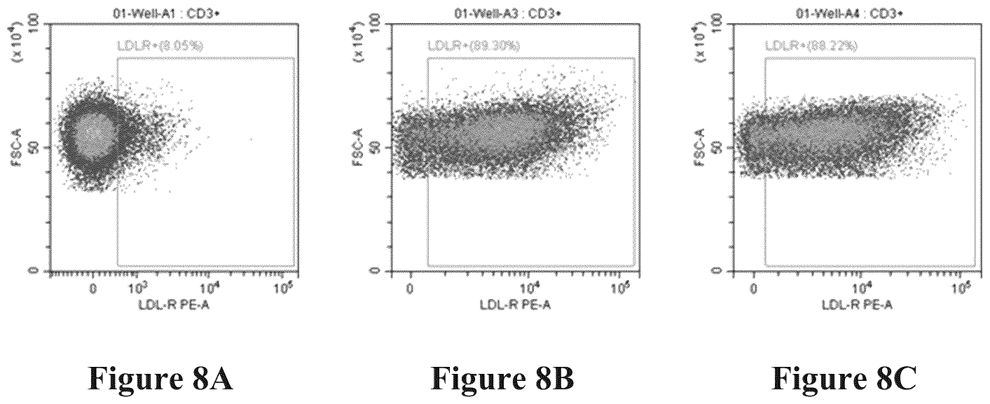

FIG. 8A is flow cytometry data showing cell-surface LDL-R expression of T cells before (Day 0) undergoing the method for upregulating low-density lipoprotein receptor (LDL-R) expression described herein and above.

The percentage of cells showing detectable levels of cell surface LDL-R expression for non-activated T cells in a pre-treatment population was 8.05%. Data was collected on Day 0.

FIGS. 8B and 8C are flow cytometry data showing cell-surface LDL-R expression on T cells having undergone (Day 1) the method for upregulating low-density lipoprotein receptor (LDL-R) expression on the cell surface described herein. The cell populations shown in FIGS. 8B and 8C have shifted to the right-hand side of their plots relative to the population shown in FIG. 8A which indicates an increase in LDL-R expression levels.

The percentage of cells showing detectable levels of cells surface LDL-R expression in a post-treatment population in Example 3, Replicate #1 was 89.30%. The percentage of cells showing detectable levels of cells surface LDL-R expression in a post-treatment population in Example 3, Replicate #2 was 88.22%. Data was collected on Day 1.

While preferred embodiments have been shown and described herein, it will be obvious to those skilled in the art that such embodiments are provided by way of example only. Numerous various, changes, and substitutions will now occur to those skilled in the art. It should be understood that various alternatives to the embodiments described herein may be employed in practice. It is intended that the following claims define the scope of the methods within the scope of these claims and their equivalents.

### INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

The foregoing written specification is considered to be sufficient to enable one skilled in the art to practice the invention. The foregoing description and Examples that follow detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

1. A method for upregulating low-density lipoprotein receptor (LDL-R) expression in a population of T cells, comprising:
providing a population of selected T cells expressing a first quantity of membrane-bound LDL-Rs; and
incubating the population of T cells in a cell culture solution comprising IL-2,
wherein the cultured population of T cells express a second quantity of membrane-bound LDL-Rs,
wherein the second quantity is larger than the first quantity.

2. The method for upregulating low-density lipoprotein receptor (LDL-R) expression in a population of T cells of claim 1, wherein the cell culture solution is 37° C.

3. The method for upregulating low-density lipoprotein receptor (LDL-R) expression in a population of T cells of claim 2, wherein the cell culture solution is maintained at a 5% CO₂ concentration.

4. The method for upregulating low-density lipoprotein receptor (LDL-R) expression in a population of T cells of claim 3, wherein the step of incubating requires at least 5 hours.

5. The method for upregulating low-density lipoprotein receptor (LDL-R) expression in a population of T cells of claim 3, wherein the step of incubating requires between about 20 hours to about 24 hours.

6. The method for upregulating low-density lipoprotein receptor (LDL-R) expression in a population of T cells of claim 1, wherein the second quantity has increased by between about 4.7-fold to about 11.0-fold compared to the first quantity.

7. The method for upregulating low-density lipoprotein receptor (LDL-R) expression in a population of T cells of claim 1, wherein the second quantity has increased by between about 4.7-fold to about 6.5-fold compared to the first quantity.

8. The method for upregulating low-density lipoprotein receptor (LDL-R) expression in a population of T cells of claim 1, wherein the second quantity has increased by between about 6.5-fold to about 11.0-fold compared to the first quantity.

9. The method for upregulating low-density lipoprotein receptor (LDL-R) expression in a population of T cells of claim 1, wherein the second quantity has increased by about 7.4-fold compared to the first quantity.

10. The method for upregulating low-density lipoprotein receptor (LDL-R) expression in a population of T cells of claim 1, wherein the population of T cells are non-activated.

11. The method for upregulating low-density lipoprotein receptor (LDL-R) expression in a population of T cells of claim 10, further comprising transducing the cultured population of non-activated T cells.

12. A method of manufacturing CAR T cells, comprising:
conducting an apheresis on a patient;
selecting a population of T cells from the apheresis;
upregulating cell surface expression of low-density lipoprotein receptors (LDL-R) of the population of T cells;
transducing the population of T cells to generate a population of CAR T cells;
causing the CAR T cells to proliferate; and
generating a cell therapy product using the CAR T cells.

13. The method of manufacturing CAR T cells of claim 12, further comprising:
cryopreserving the selected population of T cells.

14. The method of manufacturing CAR T cells of claim 13, further comprising:
thawing the cryopreserved population of T cells.

15. The method of manufacturing CAR T cells of claim 14, further comprising:
washing the thawed population of T cells.

16. The method of manufacturing CAR T cells, wherein the step of upregulating cell surface expression of low-density lipoprotein receptors (LDL-R) of the population of T cells further comprises:
incubating the population of T cells in a cell culture solution comprising IL-2.

17. The method of manufacturing CAR T cells of claim 16, wherein the cell culture solution is incubated at a temperature of 37° C.

18. The method of manufacturing CAR T cells of claim 16, wherein the cell culture solution is incubated in an environment of 5% CO₂.

19. The method of manufacturing CAR T cells of claim 16, wherein the incubation step for LDL-R upregulation requires at least 5 hours.

20. The method of manufacturing CAR T cells of claim 16, wherein the incubation step for LDL-R upregulation requires between about 20 hours to about 24 hours.

21. The method of manufacturing CAR T cells of claim 12, wherein the population of T cells are non-activated.

22. The method of manufacturing CAR T cells of claim 12, wherein the transduction step uses a lentiviral vector.

23. The method of manufacturing CAR T cells of claim 12, wherein the transductions step uses a retroviral vector.

24-31. (canceled)
